(19) United States
(12) Patent Application Publication
(10) Pub. No.: US 2006/0216697 A1
(43) Pub. Date: Sep. 28, 2006

(54) Method of separating unattached Raman-active tag from bioassay or other reaction mixture

(75) Inventors: Frank John Mondello, Niskayuna, NY (US); Michael Craig Burrell, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/087,419

(22) Filed: Mar. 24, 2005

(51) Int. Cl. C12Q 1/70; C12Q 1/68; G01N 33/551

(52) U.S. Cl. 435/5; 436/524; 435/6; 977/900; 977/924

(57) ABSTRACT

A super-paramagnetic Raman-active complex that includes a Raman-active tag attached to a target and a super-paramagnetic bead attached to the target is disclosed. A method of separating a Raman or surface enhanced Raman-active tag unattached to a target from a Raman-active complex is also disclosed. The Raman-active complex includes a Raman-active tag attached to a target. The method includes providing a mixture that includes at least one Raman-active tag unattached to a target and at least one super-paramagnetic Raman-active complex; and applying a magnetic field to the mixture.

### BACKGROUND OF THE INVENTION

The invention relates to Raman and surface enhanced Raman-active bioassays or other reaction mixture. Particularly, the invention is directed to a method of separating an unattached Raman-active tag from a bioassay or other reaction mixture.

### DESCRIPTION OF RELATED ART

Raman and surface enhanced Raman-active tags 100 are known to detect the presence of pathogenic organisms or other materials. FIG. 1 is a schematic representation of a Raman-active tag 100 that includes a Raman-active particle 110 and one or more target-binding moieties 112. The target-binding moiety 112 on the Raman-active tag 100 is configured to allow the Raman-active tag 100 to attach to one or more targets 212 to form a Raman-active complex 200. In contrast, the Raman-active tag 100 is unattached to a target 212. FIG. 2 and FIG. 2a are schematic representations of a Raman-active complex 200 comprising a Raman-active tag 100 and a target 212. In the presence of a target 212, one or more target-binding moieties 112 against a target 212 allow the Raman-active tag 100 to attach to the target 212. Detection of the target 212 is then based on the presence of a Raman signal after removing any Raman-active tags 100 that are unattached to a target 212 from the test mixture. Failure to eliminate unattached Raman-active tags 100 results in false positive detection of the presence of the target 212. Centrifugation is a method commonly used to separate unattached Raman-active tags 100 from Raman active complexes 200 that are attached to a target; however, centrifugation is inefficient because the Raman-active tags 100 have a density such that the Raman-active tags 100 pellet along with the Raman active complexes 200 and targets 212.

Thus, a need still remains for a method of separating unattached Raman-active tag 100 from bioassay or other reaction mixture.

### SUMMARY

The purpose and advantages of embodiments of the invention will be set forth and apparent from the description that follows, as well as will be learned by practice of the embodiments of the invention. Additional advantages will be realized and attained by the methods and systems particularly pointed out in the written description and claims hereof, as well as from the appended drawings. An embodiment of the invention provides a super-paramagnetic Raman-active complex. The super-paramagnetic Raman-active complex includes a Raman-active tag attached to a target and a super-paramagnetic bead attached to the target.

A second embodiment provides a method of separating a Raman-active tag unattached to a target from a Raman-active complex. The Raman-active complex includes a Raman-active tag attached to a target. The method includes (i) providing a mixture comprising at least one Raman-active tag unattached to a target and at least one super-paramagnetic Raman-active complex and (ii) applying a magnetic field to the mixture.

A third embodiment provides a method of separating a surface-enhanced Raman-active tag unattached to a target from a Raman-active complex. The method includes (i) providing a mixture comprising at least one Raman-active tag unattached to a target and at least one super-paramagnetic Raman-active complex and (ii) applying a magnetic field to the mixture. The Raman-active complex includes a Raman-active tag attached to a target. The Raman-active tag includes a Raman-active particle and a target-binding moiety comprising an antibody

The accompanying figures, which are incorporated in and constitute part of this specification, are included to illustrate and provide a further understanding of the method and system of the invention. Together with the description, the drawings serve to explain the principles of the invention.

### BRIEF DESCRIPTION OF THE DRAWINGS

FIG. 1 is a schematic representation of an unattached Raman-active tag in accordance with an embodiment of the invention;

FIG. 2 is a schematic representation of a Raman-active complex in accordance with an embodiment of the invention;

FIG. 2a is another schematic representation of Raman-active complex in accordance with an embodiment of the invention;

FIG. 3 is a schematic representation of a super-paramagnetic Raman-active complex in accordance with an embodiment of the invention;

FIG. 4 is a schematic representation of a method of separating a Raman-active tag unattached to a target from a Raman-active complex in accordance with an embodiment of the invention;

FIG. 5 is another schematic representation of a method of separating a Raman-active tag unattached to a target from a Raman-active complex in accordance with an embodiment of the invention; and

FIG. 6 is a flow chart of a method of separating a Raman-active tag unattached to a target from a Raman-active complex in accordance with an embodiment of the invention.

### DETAILED DESCRIPTION OF EXEMPLARY EMBODIMENTS

Reference will now be made in detail to exemplary embodiments of the invention, which are illustrated in the accompanying figures and examples. Referring to the drawings in general, it will be understood that the illustrations are for the purpose of describing a particular embodiment of the invention and are not intended to limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of substituents and/or variables are permissible only if such combinations result in stable compounds.

With reference to FIG. 3, there is shown one embodiment of a super-paramagnetic Raman-active complex 300. The super-paramagnetic Raman-active complex 300 includes one or more Raman-active tags 100 attached to a target 212 and one or more super-paramagnetic beads 310 attached to the target 212. As use herein, Raman includes Raman and surface enhanced Raman spectroscopy. Examples of super-paramagnetic beads 310 include, but are not limited to, nano or micron sized beads that are attracted by a magnetic field but retain no residual magnetism when the filed is removed. The super-paramagnetic beads 310 may be of any shape or size, such as but are not limited to, nano or micron sized.

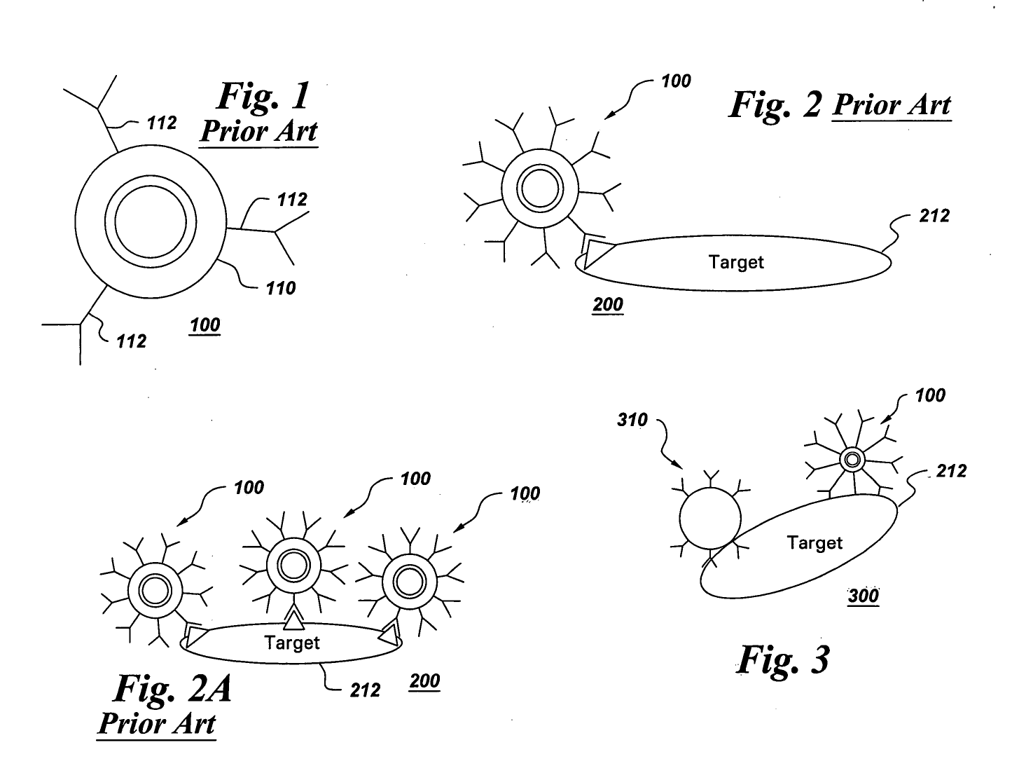

In one embodiment, the Raman-active tag 100 is immuno-functionalized. Immuno-functionalized Raman-active tags 100 detect the presence of one or more targets 212 that are pathogenic organisms or other materials. Immuno-functionalized Raman-active tags 100 include Raman-active tags 100 attached to one or more target-binding moieties 112 that are antibodies. The target-binding moiety 112 is configured to allow the Raman-active tag 100 to attach to a target 212 to form a Raman-active complex 200. Attached means the target-binding moiety 112 is covalently or non-covalently connected to a target 212. Examples of other target-binding moieties 112 include, but are not limited to, antibodies, aptamers, polypeptides, nucleic acid, peptide nucleic acids, avidin, streptavidin, and derivatives of avidin and streptavidin. The Raman-active tag 100 may comprise one target-binding moiety 112 or a plurality of target-binding moieties 112, as in FIG. 1. The plurality of target-binding moieties 112 may all be of the same kind of target-binding moieties 112 or different kinds of target-binding moieties 112.

Examples of targets 212 to which a target-binding moiety 112 may attach include, but are not limited to, organisms such as viruses, bacteria, yeast, spores, liposomes, proteins, polypeptides, toxins, nucleic acids, and beads. Examples of beads include, but are not limited to, latex, polystyrene, silica and plastic. In one embodiment, a target 212 is attached to one Raman-active complex 200 as in FIG. 2 or a plurality of Raman-active complexes 200 as in FIG. 3. In another embodiment, target-binding moieties 112 include antibodies and targets 212 include bacteria.

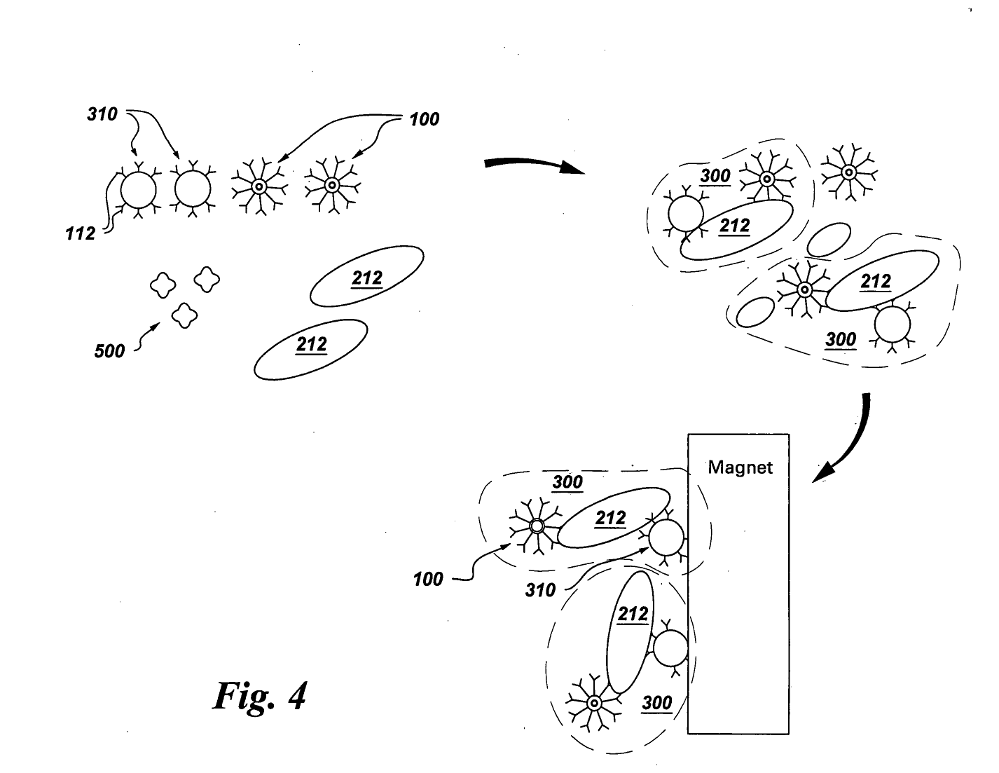

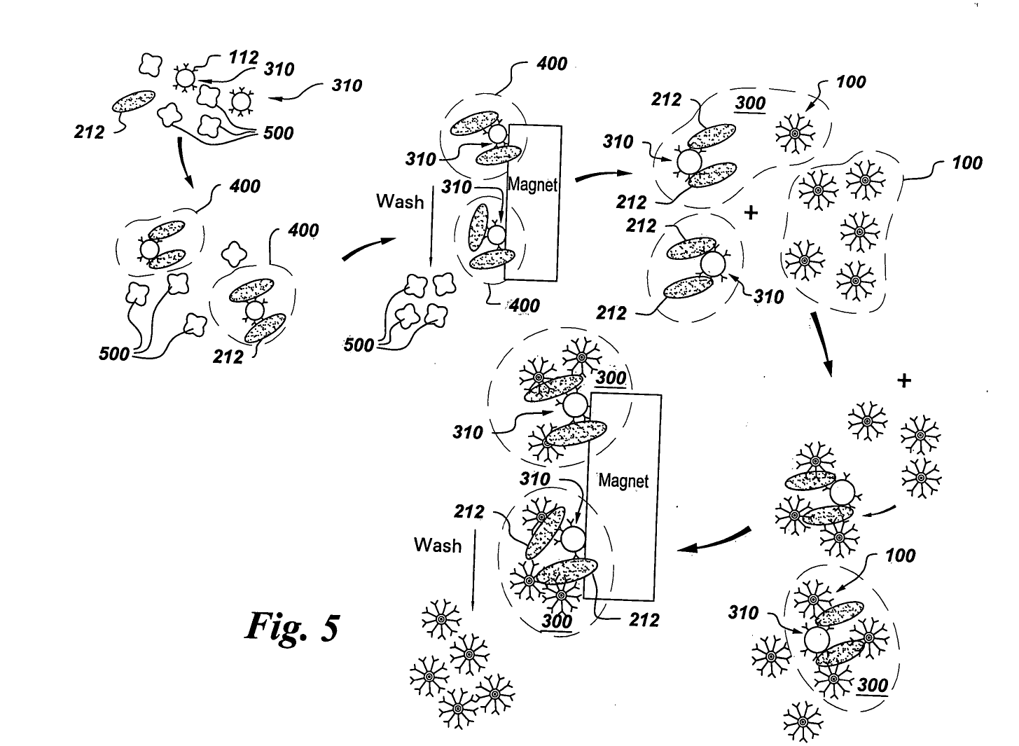

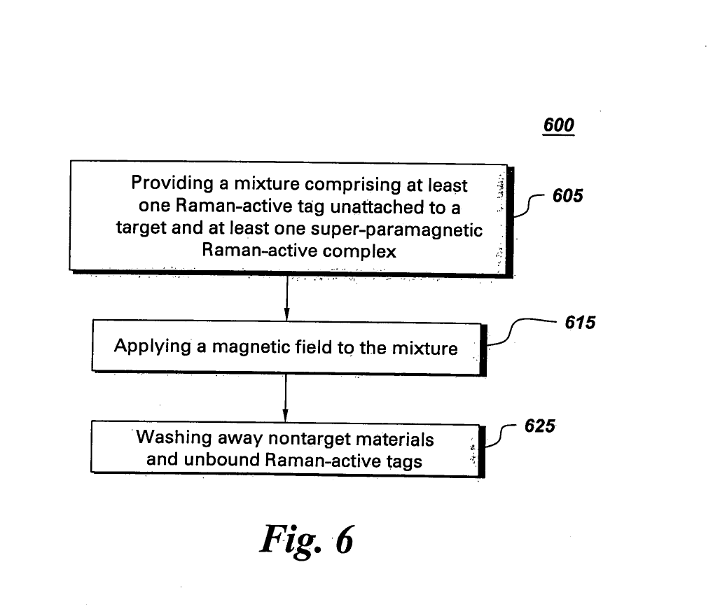

With reference to FIG. 4-FIG. 6, next will be described a method of separating one or more Raman-active tags 100 which are unattached to a target 212 from one or more Raman-active complexes 200. As previously stated, Raman includes Raman and surface enhanced Raman spectroscopy. FIGS. 4 and 5 are schematic representations of methods of separating one or more Raman-active tags 100 unattached to a target 212 from one or more Raman-active complexes 200. FIG. 6 is a flow chart of a method of separating one or more Raman-active tags 100 unattached to a target 212 from one or more Raman-active complexes 200.

As described in FIG. 6, the method includes, at Step 605, of providing a mixture comprising one or more Raman-active tags 100, and one or more super-paramagnetic Raman-active complexes 300 as described above. The mixture may also include other non-target components 500, such as impurities, toxins, etc.

In one embodiment of providing the super-paramagnetic Raman-active complexes 300, the super-paramagnetic Raman-active complexes 300 are provided by providing one or more super-paramagnetic beads 310, one or more Raman-active tags 100, and one or more targets 212. The super-paramagnetic beads 310, Raman-active tags 100, and targets 212 attach together to form the super-paramagnetic Raman-active complexes 300. The method is not limited by how the super-paramagnetic beads 310, Raman-active tags 100, and targets 212 attach. Examples of attaching include, but are not restricted to, electrostatically, chemically, and physically. The super-paramagnetic beads 310 and target may also attach together to form a super-paramagnetic-target complex 400.

The super-paramagnetic beads 310, Raman-active tags 100, and targets 212 may be provided simultaneously, as in FIG. 4, or sequentially as in FIG. 5. When providing the super-paramagnetic beads 310, Raman-active tags 100, and targets 212 sequentially, the order of providing the super-paramagnetic beads 310, Raman-active tags 100, and targets 212 may be varied. For example, in one embodiment, the super-paramagnetic beads 310 and the targets 212 are provided before the Raman-active tags 100 as in FIG. 5. In another embodiment, the Raman-active tags 100 and targets 212 can be provided before the super-paramagnetic beads 310. The super-paramagnetic beads 310 form the super-paramagnetic Raman-active complexes 300 as described above. The super-paramagnetic beads 310 and target may also form a complex 400.

Next, Step 615 includes applying a magnetic field to the mixture. The magnetic field immobilizes the super-paramagnetic beads 310 as well as the super-paramagnetic Raman-active complex 300 which comprises the super-paramagnetic beads 310, the target 212, and Raman-active tags 100. In one embodiment of applying a magnetic field, the super-paramagnetic beads 310 are in a range from about 10 nm to about 10 microns. In another embodiment of applying a magnetic field, the super-paramagnetic beads 310 are in a range from about 0.3 micron to about 1.5 microns.

The method may also further comprise taking a Raman spectrum of the super-paramagnetic Raman-active complex 300. The Raman spectrum may be taken directly after a washing Step 625 to remove any unattached Raman-active tags 100 and other non-target 500 components of the mixture that are in solution. The super-paramagnetic Raman-active complexes 300 are then removed from the magnetic field and resuspended in a small volume of buffer to take the Raman spectrum.

The following example serves to illustrate the features and advantages of the invention and is not intended to limit the invention thereto.

Magnetic Particle Method Example (Generalized):

A sample of target microorganisms 212, which includes but is not restricted to bacteria, spores, and viruses, is added to a sample container such as an eppindorf tube.

A quantity of nanometer or micrometer sized super-paramagnetic (SPR) beads 310 attached to antibodies against the target microorganism 212 are added to the sample.

A quantity of Raman-active tags 100 attached to antibodies against the target microorganism 212 is added to the sample.

The sample is mixed and incubated at room temperature for a period of time.

The mixture is placed in a magnetic field. The magnetic field immobilizes the SPR particles 310, as well as the super-paramagnetic Raman-active complex 300 which comprises the SPR bead 310, the target 212, and Raman-active tags 100. The magnetic field immobilizes the SPR bead and the super-paramagnetic Raman-active complex 300 onto the wall of the tube 310.

Unattached Raman-active tags and other components of the mixture remain in solution and are removed by washing.

After washing, the super-paragmagnetic Raman-active complex 300 (i.e. SPR-Target-Raman-active complexes) are removed from the magnetic field and resuspended in a small volume of buffer.

A portion of the buffer is then analyzed for the presence of a Raman-active signal.

Thus, Example 1 demonstrates how it is possible to use immuno-functionalized Raman-active tags 100 to detect the presence of a specific target organism 212. In these experiments, a Raman signal is only detected when the appropriate target organism 212 and Raman-active tags 100 immuno-functionalized for that specific target organism 212 to detect the presence of that specific target organism 212 are both present.

While the invention has been described in detail in connection with only a limited number of aspects, it should be readily understood that the invention is not limited to such disclosed aspects. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method of separating a Raman-active tag unattached to a target from a Raman-active complex, wherein the Raman-active complex comprises a Raman-active tag attached to a target,
the method comprising:
i) providing a mixture comprising at least one Raman-active tag unattached to a target and at least one super-paramagnetic Raman-active complex; and
ii) applying a magnetic field to the mixture.

2. The method of claim 1, wherein providing the at least one super-paramagnetic Raman-active complex comprises providing a super-paramagnetic bead, a Raman-active tag, and a target.

3. The method of claim 2, wherein the super-paramagnetic bead, Raman-active tag and a target are simultaneously provided.

4. The method of claim 2, wherein the super-paramagnetic bead, Raman-active tag, and target are sequentially provided.

5. The method of claim 3, wherein the super-paramagnetic bead, and the target are provided before the Raman-active tag.

6. The method of claim 4, wherein the Raman-active tag and the target are provided before the super-paramagnetic bead.

7. The method of claim 1, wherein applying the magnetic field comprises applying a sufficient magnetic field to separate the at least one Raman-active tag unattached to a target from the at least one Raman-active complex.

8. The method of claim 1, wherein the Raman-active tag comprises a Raman-active particle and a target-binding moiety, wherein the target-binding moiety is configured to attach the Raman-active particle to a target.

9. The method of claim 8, wherein the target-binding moiety comprises at least one chemical moiety selected from a group consisting of antibodies, aptamers, nucleic acid, and polypeptides.

10. The method of claim 1, further comprising taking a Raman spectrum of the super-paramagnetic Raman-active complex.

11. The method of claim 1, wherein the target comprises at least one target selected from a group consisting of viruses, bacteria, proteins, polypeptides, toxins, nucleic acids, and spores.

12. The method of claim 1, wherein the target is attached to a plurality of Raman-active complexes.

13. The method of claim 1, wherein the super-paramagnetic bead is in a range from about 10 nm to about 10 microns.

14. The method of claim 13, wherein the super-paramagnetic bead is in a range from about 0.3 microns to about 1.5 microns.

15. A method of separating a surface enhanced Raman-active tag unattached to a target from a Raman-active complex, wherein the Raman-active complex comprises a Raman-active tag attached to a target,
the method comprising:
i) providing a mixture comprising at least one Raman-active tag unattached to a target and at least one super-paramagnetic Raman-active complex; wherein the Raman-active tag comprises a Raman-active particle and a target-binding moiety comprising an antibody; and
ii) applying a magnetic field to the mixture.

16. The method of claim 15, wherein applying the magnetic field comprises applying a sufficient magnetic field to separate the Raman-active tag unattached to a target from a Raman-active complex.

17. The method of claim 15, wherein the super-paramagnetic bead is in a range from about 10 nm to about 10 microns.

18. The method of claim 17, wherein the super-paramagnetic bead is in a range from about 0.3 microns to about 1.5 microns.

19. The method of claim 15, further comprising taking a Raman spectrum of the Raman-active complex.

20. The method of claim 15, wherein the target comprises at least one target selected from a group consisting of viruses, bacteria, proteins, polypeptides, toxins, nucleic acids, and spores.

21. The method of claim 15, wherein the target is attached to a plurality of Raman-active complexes.

22. A super-paramagnetic Raman-active complex comprising:
(i) a Raman-active tag attached to a target; and
(ii) a super-paramagnetic bead attached to the target.

23. The super-paramagnetic Raman-active complex of claim 22, wherein the Raman-active tag comprises a Raman-active particle and a target-binding moiety, wherein the target-binding moiety is configured to attach the Raman-active particle to the target.

24. The super-paramagnetic Raman-active complex of claim 23, wherein the target-binding moiety comprises at least one chemical moiety selected from a group consisting of antibodies, aptamers, nucleic acid, and polypeptides.

25. The super-paramagnetic Raman-active complex of claim 22, wherein the target comprises at least one target selected from a group consisting of viruses, bacteria, proteins, polypeptides, toxins, nucleic acids, and spores.

26. The super-paramagnetic Raman-active complex of claim 22, wherein the target is attached to a plurality of Raman-active complexes.

27. The super-paramagnetic Raman-active complex of claim 22, wherein the target is attached to a plurality of super-paramagnetic beads.
